(19) United States
(12) Patent Application Publication
(10) Pub. No.: US 2026/0151083 A1
(43) Pub. Date: Jun. 4, 2026

(54) CONNECTED FORMULA IDENTIFIABLE BY DEVICE LOW COST SENSOR TO CUSTOMIZE TREATMENT

(71) Applicant: L'OREAL, Paris (FR)

(72) Inventors: Gregoire Charraud, Jersey City, NJ (US); Rafael Feliciano, New Providence, NJ (US)

(73) Assignee: L'OREAL, Paris (FR)

(21) Appl. No.: 18/968,104

(22) Filed: Dec. 4, 2024

(51) Int. Cl. A61B 5/00; A61B 5/01; A61P 17/00

(52) U.S. Cl. CPC A61B 5/4848; A61B 5/0075; A61B 5/01; A61B 5/442; A61B 5/746; A61P 17/00

(57) ABSTRACT

A system for applying and analyzing a skin treatment formula is applied herein. The system may include at least one topical skin treatment formula and a skin treatment sensor device. The skin treatment sensor device may include one or more sensors and a skin treatment effectiveness unit. The skin sensors may be configured to collect a plurality of skin surface measurements. The skin treatment effectiveness unit may include a processor, a memory unit, and a user alert unit. The processor may be configured to identify a type and an amount of topical skin treatment formula applied to the skin surface based on the skin surface measurements. The memory unit may be configured to store a plurality of reference values and user baseline measurements related to the topical skin treatment formula and the skin surface. The user alert unit may be configured to present one or more notifications to a user.

### SUMMARY

Disclosed herein is a system for applying and analyzing a skin treatment formula, including at least one topical skin treatment formula and a skin treatment sensor device. The topical skin treatment formulas may be configured for applying to a skin surface. The skin treatment sensor device may include one or more skin sensors and a skin treatment effectiveness unit. The one or more skin sensors may be configured to collect a plurality of skin surface measurements. The skin treatment effectiveness unit may be electrically coupled with one or more skin sensors and may include a processor, a memory unit, and a user alert unit. The processor may be configured to identify a type and an amount of topical skin treatment formula applied to the skin surface based on the plurality of skin surface measurements. The memory unit may be electrically coupled with the processor and configured to store a plurality of reference values corresponding to the type and amount of the topical skin treatment formula applied to the skin surface and a plurality of user baseline measurements corresponding to the skin surface. The user alert unit may be configured to present one or more notifications to a user.

In some embodiments, the one or more skin sensors includes an electroconductivity sensor, a spectroscopy sensor, and/or a temperature sensor.

In some embodiments, the plurality of baseline measurements, the plurality of skin surface measurements, and the plurality of reference values includes skin resistivity, skin reflectivity, and/or skin temperature.

In some embodiments, the processor is configured to compare the plurality of baseline measurements with the plurality of skin surface measurements and the plurality of reference values. Based on this comparison, the processor may also be configured to determine which notification to present to the user.

In some embodiments, notifications includes a notification directing the user to apply the skin treatment formula to the skin surface and a notification directing the user to cease treatment.

In some embodiments, the skin treatment sensor device also includes a power storage source configured to independently power the skin treatment sensor device.

In some embodiments, the skin treatment sensor device also includes a formula dispensing mechanism, which may be electrically coupled to the formula dispensing mechanism.

In another aspect, disclosed herein is a skin treatment sensor device including one or more skin sensors and a skin treatment effectiveness unit. The skin sensors may be configured to collect a plurality of skin surface measurements. The skin treatment effectiveness unit may be electrically coupled with the one or more skin sensors and may include a processor, a memory unit, and a user alert unit. The processor may be configured to identify a type and an amount of at least one topical skin treatment formula applied to a skin surface based on the plurality of skin surface measurements. The memory unit may be electrically coupled with the processor and may be configured to store a plurality of reference values corresponding to the type and amount of the topical skin treatment formula applied to the skin surface and a plurality of baseline measurements corresponding to the skin surface. The user alert unit is electrically coupled with the processor and configured to present one or more notifications to a user.

In some embodiments, the one or more skin sensors include an electroconductivity sensor, a spectroscopy sensor, and/or a temperature sensor.

In some embodiments, the plurality of baseline measurements, the plurality of skin surface measurements, and the plurality of reference values include skin resistivity, skin reflectivity, and/or skin temperature.

In some embodiments, the processor is configured to compare the plurality of baseline measurements with the plurality of skin surface measurements and the plurality of reference values. Based on this comparison, the processor is configured to determine which notification to present to the user.

In some embodiments, the one or more notifications include a notification directing the user to apply the skin treatment formula to the skin surface and a notification directing the user to cease treatment.

In some embodiments, the skin treatment sensor device also includes a formula dispensing mechanism, which is electrically coupled to the skin treatment effectiveness unit.

In some embodiments, the skin treatment sensor device also includes a power storage source configured to independently power the skin treatment sensor device.

In another aspect, disclosed herein is a method for determining the effectiveness of a skin treatment including collecting a plurality of user baseline measurements; applying at least one topical skin treatment formulas to a skin surface; identifying, by a skin treatment sensor device, the at least one topical skin treatment formulas applied to the skin surface; collecting a plurality of skin surface measurements; comparing a plurality of reference values, the plurality of skin surface measurements, and the plurality of user baseline measurements; and alerting a user of the effectiveness of the treatment.

In some embodiments, the plurality of baseline measurements, the plurality of skin surface measurements, and the plurality of reference values include skin resistivity, skin reflectivity, and/or skin temperature.

In some embodiments, the skin treatment sensor device includes one or more skin sensors and a skin treatment effectiveness unit. The skin sensors may be configured to collect a plurality of skin surface measurements. The skin treatment effectiveness unit may be electrically coupled with the one or more skin sensors and may include a processor, a memory unit, and a user alert unit. The processor may be configured to identify a type and an amount of at least one topical skin treatment formula applied to a skin surface based on the plurality of skin surface measurements. The memory unit may be electrically coupled with the processor and may be configured to store a plurality of reference values corresponding to the type and amount of the topical skin treatment formula applied to the skin surface and a plurality of baseline measurements corresponding to the skin surface. The user alert unit is electrically coupled with the processor and configured to present one or more notifications to a user.

In some embodiments, the processor is configured to compare the plurality of baseline measurements with the plurality of skin surface measurements and the plurality of reference values. Based on this comparison, the processor may also be configured to determine which notification to present to the user.

In some embodiments, the one or more notifications include a notification directing the user to apply the skin treatment formula to the skin surface and a notification directing the user to cease treatment.

In some embodiments, the method also includes dispensing, by the skin treatment sensor device, a topical skin treatment formula.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1 is an example system for applying and analyzing a skin treatment formula applied to a skin surface, in accordance with the present technology.

FIGS. 2A-2C are example systems for analyzing a skin treatment formula applied to a skin surface, in accordance with the present technology.

FIGS. 3A-3B are example formula dispensing mechanisms for a skin treatment sensor device, in accordance with the present technology.

FIGS. 4A-4B are example methods for using a system for applying and analyzing a skin treatment formula with a skin treatment sensor device, in accordance with the present technology.

The foregoing aspects and many of the attendant advantages of this invention will become more readily appreciated as the same become better understood by reference to the following detailed description, when taken in conjunction with the accompanying drawings.

### DETAILED DESCRIPTION

Examples of a system, apparatus, and method for applying and analyzing a skin treatment formula are disclosed herein. Thus, in the following description, numerous specific details are set forth to provide a thorough understanding of the examples. One skilled in the relevant art will recognize; however, that the techniques described herein can be practiced without one or more of the specific details, or with other methods, components, materials, etc. In other instances, well-known structures, materials, or operations are not shown or described in detail to avoid obscuring certain aspects.

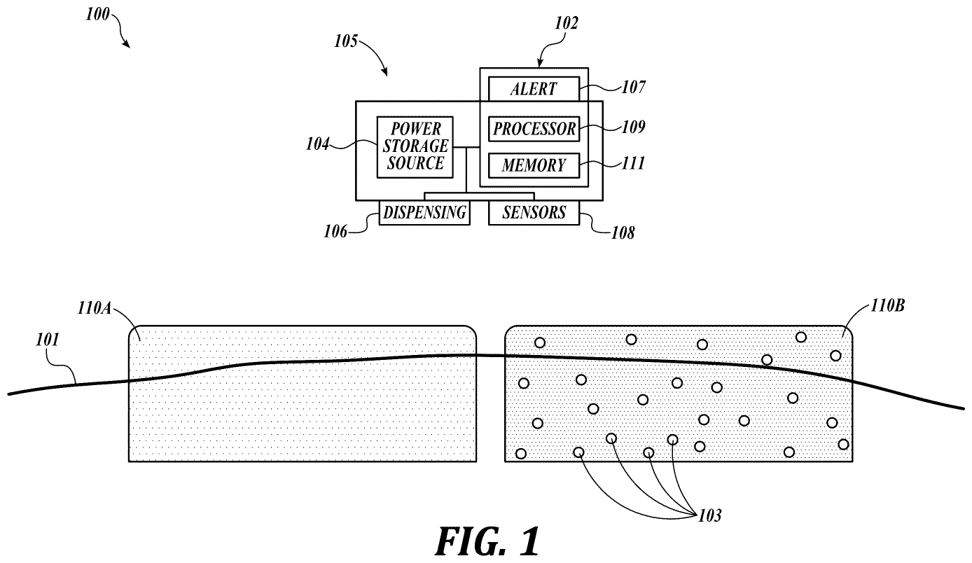

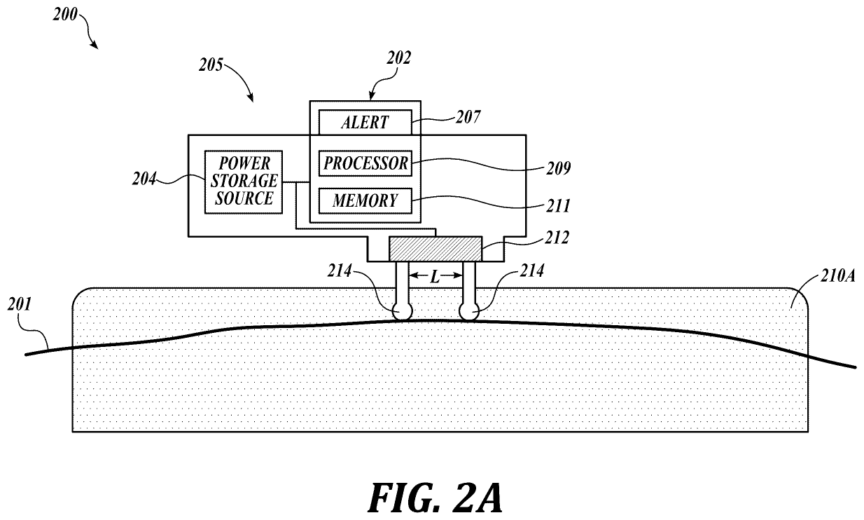

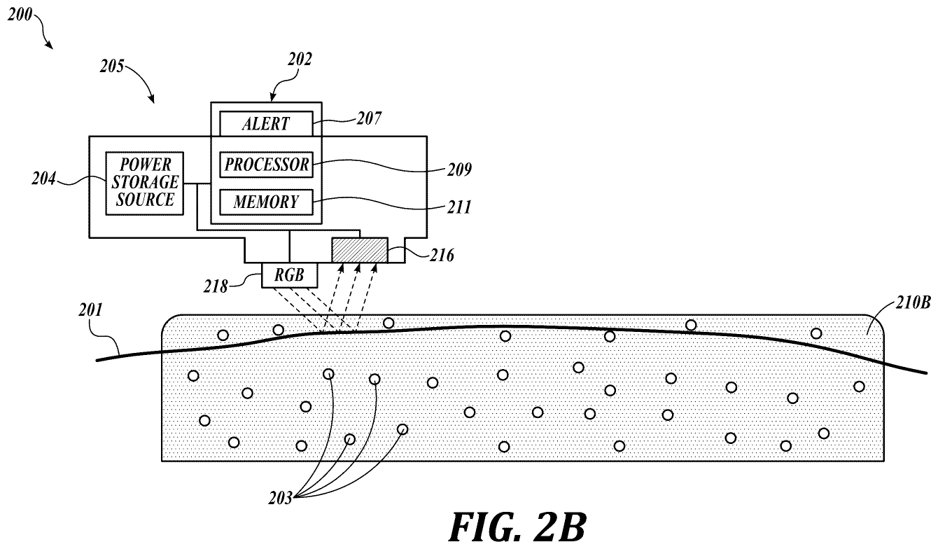

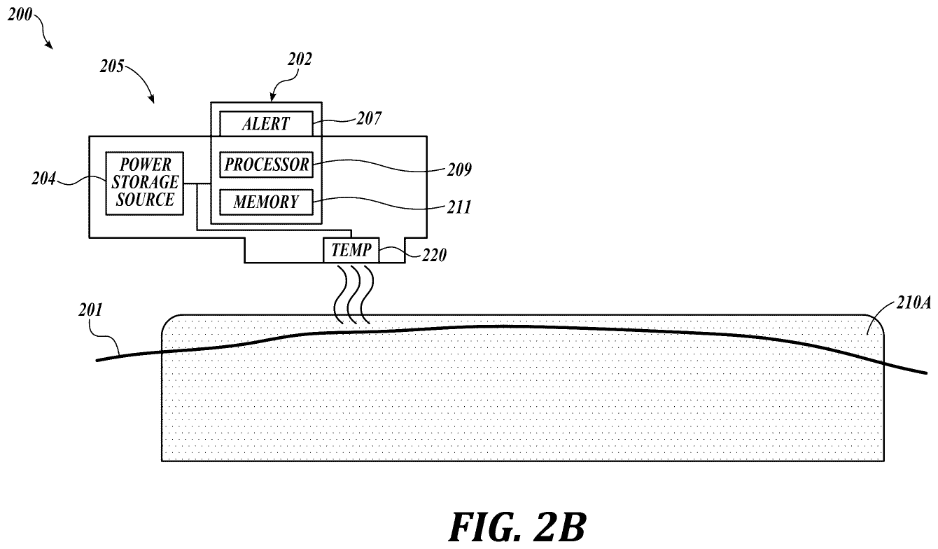

FIG. 1 is an example system for applying and analyzing a skin treatment formula applied to a skin surface, in accordance with the present technology. In some embodiments, system 100 includes at least one topical skin treatment formula 110A, 110B and a skin treatment sensor device 105. In some embodiments, the topical skin treatment formulas 110A, 110B is configured for applying to a skin surface 101. In some embodiments, the topical skin treatment formulas 110A, 110B has characteristics that may be used measure and characterize its electroconductivity, spectroscopy, and/or temperature. In the illustrated example, the topical skin treatment formula 110B includes reflective particles 103 (e.g., gold particles) to characterize the spectroscopy of the formula 110B. In some embodiments, the skin treatment sensor device 105 includes one or more skin sensors 108, including an electroconductivity sensor, a spectroscopy sensor, and/or a temperature sensor. In such embodiments, these sensors 108 are configured to collect a plurality of skin surface measurements, including skin resistivity, skin reflectivity, and skin temperature. As further described below, these skin surface measurements are influenced by the presence of the topical skin formulas 110A, 110B, thus providing a relationship of the skin surface measurements versus type and quantity of the skin formulas 110A, 110B applied at different locations of the skin surface 101. These sensors 108 and their respective measurements are described in greater detail in FIGS. 2A-2C.

In some embodiments, the skin treatment sensor device also includes a skin treatment effectiveness unit 102, which is electrically coupled with the one or more skin sensors 108 and includes a user alert unit 107, a processor 109, and a memory unit 111. In some embodiments, the processor is configured to identify a type and an amount of the topical skin treatment formulas 110A, 110B applied to the skin surface 101. In such embodiments, the processor 108 is configured to make such determinations based on the plurality of skin measurements collected by the one or more skin sensors 108 and the relevant characteristics (e.g., electroconductivity, optical properties, and/or temperature) of the topical skin treatment formulas 110A, 110B. In some embodiments, the memory unit 111 is electrically coupled to the processor 109 and configured to store a plurality of user baseline measurements, which may include initial skin surface measurements (e.g., skin resistivity, skin reflectivity, skin temperature). These user baseline measurements may be collected by the skin sensor device 105 on a user's untreated skin surface 101 upon initial or subsequent uses of the device 105. The memory unit 111 may also be configured to store a plurality of reference values corresponding to the type and amount of the topical skin treatment formula 110A, 110B applied to the skin surface 101. The reference values may include known and quantifiable skin surface measurements that result from the unique characteristics of the topical skin treatment formulas 110A, 110B having been applied to the skin surface, as well as desirable ranges or values for such skin surface measurements. The memory unit 111 may also be configured to store a plurality of skin surface measurements. For example, in an instance where a user applies topical skin treatment formula 110B, a memory unit 111 may store a measurement of the user's untreated skin's reflectivity (a baseline measurement), experimental data concerning the quantifiable effect of topical skin treatment formula 110B on a user's skin reflectivity (a reference value), a desired (i.e., ideal) range for skin reflectivity when treated by topical skin treatment formula 110B, and a measurement of the user's treated skin resistivity (a skin surface measurement).

In some embodiments, the user alert unit 107 is electrically coupled to the processor 109 and is configured to present one or more notifications to a user. In some embodiments, the user alert unit 107 includes a speaker. In such embodiments, the one or more notifications may be auditory and comprise one or more pre-recorded tones or, alternatively, a pre-recorded voice message. In some embodiments, the user alert unit 107 includes a digital screen. In such embodiments, the one or more notifications may be visual and take the form of text displayed on the digital screen. In some embodiments, the user alert unit includes one or more LEDs. In such embodiments, the one or more notifications may be visual and take the form of one or more LEDs being illuminated. In some embodiments, these notifications include a notification directing the user to apply the at least one topical skin treatment formula 110A, 110B to the skin surface 101, and/or a notification directing the user to cease treatment. In some embodiments, the processor 109 is configured for comparing the plurality of skin surface measurements made by the one or more sensors 106, the plurality of reference values stored in the memory unit 111, and the plurality of user baseline measurements made by the one or more sensors 106 and stored in the memory unit 111. In such embodiments, the processor 109 is further configured to determine which of the one or more notifications to present to the user based upon this comparison direct the user alert unit 107 to present to the user.

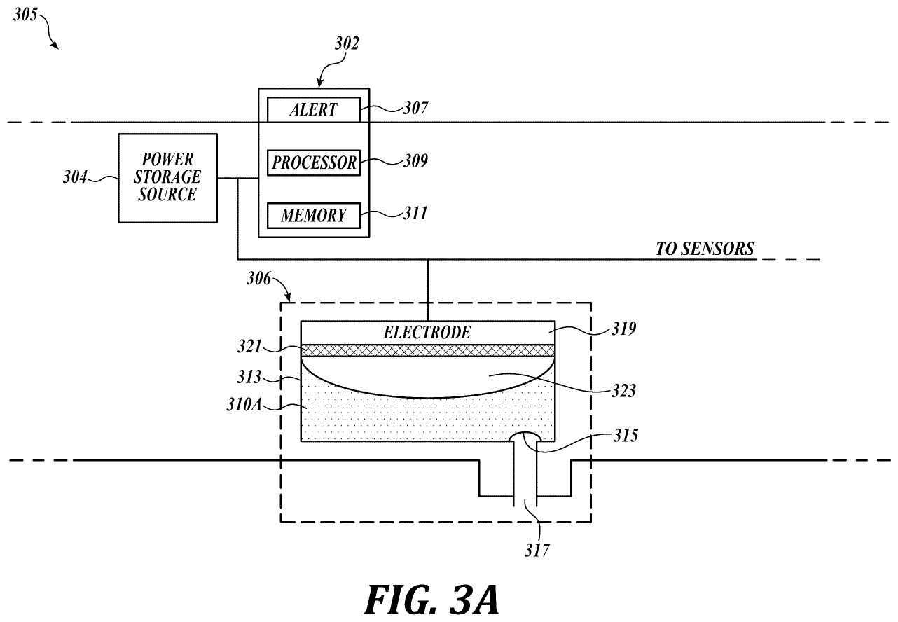

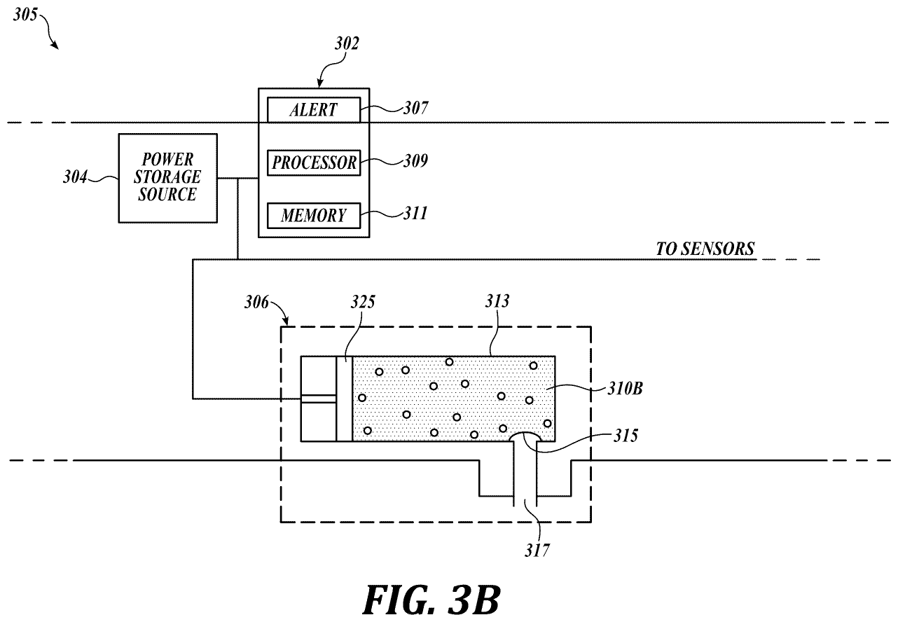

In some embodiments, the skin treatment sensor device 105 further includes a formula dispensing mechanism 106, which is electrically coupled with the skin treatment effectiveness unit 102 and configured to dispense a topical skin treatment formula 110A, 110B on to a skin surface 101. In some embodiments, the formula dispensing mechanism 106 dispenses a topical skin treatment formula 110A, 110B using a piezoelectric dispensing mechanism (as described in FIG. 3A below). In some embodiments, the formula dispensing mechanism 106 dispenses a topical skin treatment formula 110A, 110B using an actuator (as described in FIG. 3B below). Example dispensing mechanisms are discussed in greater detail in FIGS. 3A-3B.

In some embodiments, the skin treatment sensor device 105 further includes a power storage source 104, which is electrically coupled with the skin treatment effectiveness unit 102 and its subcomponents (e.g., user alert unit 107, processor 109, and memory unit 111), the formula dispensing mechanism 106, and the one or more sensors 108. In some embodiments, the power storage source 104 is configured to independently power the skin treatment device 105. In some embodiments, the power storage source 104 is a battery or a rechargeable battery. The power storage source 104 can be charged by power delivered directly by a cable connected to the appliance (not shown). In an alternative embodiment the power storage source 104 can be charged by any wireless means including by pLink charging system, inductive Qi charging system and AirFuel.

FIGS. 2A-2C are example systems 200 for applying and analyzing a skin treatment formula 210A, 210B applied to a skin surface 201, in accordance with the present technology. In some embodiments, the skin treatment sensor device 205 includes one or more skin sensors, including an electroconductivity sensor 212, a spectroscopy sensor 216, and/or a temperature sensor 218.

FIG. 2A is an example device 205 including an electroconductivity sensor 212, in accordance with the present technology. In some embodiments, the electroconductivity sensor 212 is electrically coupled with the power storage source 204 and the skin treatment effectiveness unit 202, specifically the processor 209. In some embodiments, the electroconductivity sensor 212 is configured to collect a plurality of skin surface measurements, including skin resistivity. In such embodiments, a plurality of electrodes 214 are electrically coupled with the electroconductivity sensor 212 and brought into physical contact with the skin surface 201. When the plurality of electrodes 214, separated by a distance L, make physical contact with the skin surface 201, a small current is run between them through the skin surface 201, and the resistance experienced by the current is measured by the electroconductivity sensor 212.

In some embodiments, the skin resistivity measurement is made on an untreated skin surface 201, where no topical skin treatment formula 210A is applied. In such embodiments, this measurement serves as a baseline measurement, which may be stored in memory unit 211 and referenced by the processor 209 in subsequent comparisons, calculations, and logical determinations as required. In some embodiments, the measurement is made on a skin surface 201 treated by a topical skin treatment formula 210A. In the illustrated example, topical skin treatment formula 210A has characteristics that may affect and quantifiably change the resistivity of the skin surface 201 when the formula is applied. In such embodiments, the measurement serves as a characterization of the formula presence and quantity over the skin surface. In some embodiments, skin surface measurements may be used for instantaneous comparisons by the processor 209 without being stored in memory 211. In other embodiments, skin surface measurements may be stored in memory unit 211 and referenced by processor 209 as required.

In some embodiments, the memory unit 211 is configured to store a plurality of reference values corresponding to the type and amount of the topical skin treatment formula applied to a skin surface 201. The reference values may include known and quantifiable skin surface measurements that result from the unique characteristics of the topical skin treatment formulas 210A. For example, the memory unit 211 may store a measurement of the user's untreated skin's resistivity (a baseline measurement), experimental data concerning the quantifiable effect of topical skin treatment formula 210A on a user's skin resistivity (a reference value), an ideal range for treated skin resistivity (a reference value), and a measurement of the user's treated skin's resistivity (a skin surface measurement).

In some embodiments, the processor 209 is configured for comparing the plurality of skin surface measurements made by the electroconductivity sensor 212, the plurality of reference values stored in the memory unit 211, and the plurality of user baseline measurements made by the electroconductivity sensor 212 and stored in the memory unit 211. In such embodiments, the processor 209 is further configured to determine which of the one or more notifications to present to the user based upon this comparison direct the user alert unit 207 to present to the user. In some embodiments, these notifications include a notification directing the user to apply the topical skin treatment formula 210A to the skin surface 201, and/or a notification directing the user to cease treatment.

FIG. 2B is an example device 205 including a spectroscopy sensor 216 and a Red-Green-Blue (RGB) light emitting diode (LED) 218, in accordance with the present technology. In other embodiments, other sources of light 218 may be used. In some embodiments, both the spectroscopy sensor 216 and the RGB LED 218 are electrically coupled with the power storage source 204 and the skin treatment effectiveness unit 202, specifically with the processor 209. In some embodiments, the spectroscopy sensor 216 is configured to collect a plurality of skin surface measurements, including skin reflectivity. In such embodiments, the RGB LED 218 is activated, illuminating the skin surface 201. The light from the RGB LED 218 reflects off the skin surface 201, and the reflected light is captured by the spectroscopy sensor 216, which is configured to measure the reflectivity of the skin surface 201 from this reflected light.

In some embodiments, the skin reflectivity measurement is made on an untreated skin surface 201, where no topical skin treatment formula 210B is applied. In such embodiments, the measurement serves as a baseline measurement, which may be stored in memory unit 211 and referenced by processor 209 in subsequent comparisons, calculations, and logical reasoning as required. In some embodiments, the measurement is made on a skin surface 201 treated by a topical skin treatment formula 210B. In the illustrated example, the topical skin treatment formula 210B includes reflective particles 203 (e.g., gold particles) to characterize the spectroscopy of formula 210B. In such embodiments, the measurement serves as a skin surface measurement. In some embodiments, skin surface measurements may be used for instantaneous comparisons by the processor 209 without being stored in memory 211. In other embodiments, skin surface measurements may be stored in memory unit 211 and referenced by processor 209 as required.

In some embodiments, the memory unit 211 is configured to store a plurality of reference values corresponding to the type and amount of the topical skin treatment formula applied to a skin surface 201. The reference values may include known and quantifiable skin surface measurements that result from the unique characteristics of the topical skin treatment formulas 210B being present on the skin surface. For example, the memory unit 211 may store a measurement of the user's untreated skin's reflectivity (a baseline measurement), experimental data concerning the quantifiable effect of topical skin treatment formula 210B on a user's skin reflectivity (a reference value), an ideal range for treated skin reflectivity (a reference value), and a measurement of the user's treated skin's reflectivity (a skin surface measurement).

In some embodiments, the processor 209 is configured for comparing the plurality of skin surface measurements made by the spectroscopy sensor 216, the plurality of reference values stored in the memory unit 211, and the plurality of user baseline measurements made by the spectroscopy sensor 216 and stored in the memory unit 211. In such embodiments, the processor 209 is further configured to determine which of the one or more notifications to present to the user based upon this comparison direct the user alert unit 207 to present to the user. In some embodiments, these notifications include a notification directing the user to apply the topical skin treatment formula 210B to the skin surface 201, and a notification directing the user to cease treatment.

FIG. 2C is an example device 205 including a temperature sensor 218, in accordance with the present technology. In some embodiments, the temperature sensor 220 is electrically coupled with the power storage source 204 and the skin treatment effectiveness unit 202, specifically with the processor 209. In some embodiments, the temperature sensor 220 is configured to collect a plurality of skin surface measurements, including skin temperature, either by contacting the skin surface or in a contactless manner. In some embodiments, the temperature sensor 220 is a thermocouple. In some embodiments, the temperature sensor 220 is a thermistor. In some embodiments—including the illustrated example—the temperature sensor 220 is an infrared (IR) sensor.

In some embodiments, the skin temperature measurement is made on an untreated skin surface 201, where no topical skin treatment formula 210A is applied. In such embodiments, the measurement serves as a baseline measurement, which may be stored in memory unit 211 and referenced by processor 209 in subsequent comparisons, calculations, and logical reasoning as required. In some embodiments, the measurement is made on a skin surface 201 treated by a topical skin treatment formula 210A. In the illustrated example, topical skin treatment formula 210A has characteristics that may characterize and quantifiably change the temperature measurement of the skin surface 201 when applied. In such embodiments, the measurement serves as a skin surface measurement. For example, the skin treatment formulation may act as a thermal insulator that locally affects temperature of the outside surface of the user's skin surface. In some embodiments, skin surface measurements may be used for instantaneous comparisons by the processor 209 without being stored in memory 211. In other embodiments, skin surface measurements may be stored in memory unit 211 and referenced by processor 209 as required.

In some embodiments, the memory unit 211 is configured to store a plurality of reference values corresponding to the type and amount of the topical skin treatment formula applied to a skin surface 201. The reference values may include known and quantifiable skin surface measurements that result from the unique characteristics of the topical skin treatment formulas 210A. For example, the memory unit 211 may store a measurement of the user's untreated skin's temperature (a baseline measurement), experimental data concerning the quantifiable effect of topical skin treatment formula 210A on a user's skin temperature (a reference value), an ideal range for treated skin temperature (a reference value), and a measurement of the user's treated skin's temperature (a skin surface measurement).

In some embodiments, the processor 209 is configured for comparing the plurality of skin surface measurements made by the temperature sensor 220, the plurality of reference values stored in the memory unit 211, and the plurality of user baseline measurements made by the temperature sensor 220 and stored in the memory unit 211. In such embodiments, the processor 209 is further configured to determine which of the one or more notifications to present to the user based upon this comparison direct the user alert unit 207 to present to the user. In some embodiments, these notifications include a notification directing the user to apply the topical skin treatment formula 210A to the skin surface 201, and a notification directing the user to cease treatment.

FIGS. 3A-3B are example formula dispensing mechanisms 306 for a skin treatment sensor device 305, in accordance with the present technology. In some embodiments, the formula dispensing mechanism 306 dispenses a topical skin treatment formula 310A, 310B using a piezoelectric dispensing mechanism. In some embodiments, the formula dispensing mechanism 306 dispenses a topical skin treatment formula 310A, 310B using an actuator. In the illustrated embodiments, the topical skin treatment formulas 310A, 310B are described and illustrated as being dispensed individually. However, in some embodiments the topical skin treatment formulas 310A, 310B may be packed together and dispensed individually, sequentially or commonly, as appropriate, to the target surface of skin.

FIG. 3A is an example piezoelectric formula dispensing mechanism 306 for a skin treatment sensor device 305, in accordance with the present technology. In some embodiments, the formula dispensing mechanism 306 is electrically coupled with the power storage source 304 and the skin treatment effectiveness unit 302, specifically the processor 309. In some embodiments, the piezoelectric formula dispensing mechanism 306 includes a formula reservoir 313, a formula outlet 317 covered by a check valve 315, an electrode 319, a piezoelectric material 321, and a diaphragm 323. In some embodiments, the piezoelectric material 321 is a Lead Zirconate Titanate (PZT), Barium Titanate (BTO), BiScO3—PbTiO3 (BSPT) powder, or other material. In some embodiments, the diaphragm 323 is a flexible sheet of metal. In some embodiments, the piezoelectric material 321 and the diaphragm 323 are physically coupled (e.g. “sandwiched” together) to form a piezo disc actuator. In some embodiments, the electrode 319, piezoelectric material 321 and diaphragm 323 are physically coupled with the formula reservoir 313. In some embodiments, the piezoelectric material 321 and diaphragm 323 are disposed within the formula reservoir 313. In some embodiments the formula reservoir is filled with a quantity of topical skin treatment formula 310A.

In some embodiments, the electrode 319 receives power from the power storage source 304. In some embodiments, the electrode 319 applies a voltage to the piezoelectric material 321, causing it to deform. In turn, this causes the diaphragm 323 to expand or contract commensurately with the deformation. The movement of the diaphragm 323 generates a change in pressure within the formula reservoir 313, causing the formula 310A inside to flow out of the formula reservoir 313 through the outlet 317, as regulated by the check valve 315. In some embodiments, the piezo disc actuator may be excited directly via the power storage source 304 without the use of a separate electrode 319. In some embodiments, the processor 309 regulates the operations and functions of the check valve 315. In some embodiments, the rate of flow of the formula 310A can be adjusted by the processor 309 by (1) regulating the voltage applied by the electrode 319 to the piezoelectric material 321; (2) applying one or more square wave signals through pulse width modulation (PWM) to the piezoelectric material 321 to control the displacement of the diaphragm 323; or (3) a combination of the two, so as to control the speed of fluctuation of the of the piezoelectric material 321. Regulating the voltage applied controls the volume of the formula 310A dispensed, while PWM controls the formula's 310A rate of flow out of the outlet 317.

FIG. 3B is an example actuator-driven formula dispensing mechanism 306 for a skin treatment sensor device 305, in accordance with the present technology. In some embodiments, the formula dispensing mechanism 306 is electrically coupled with the power storage source 304 and the skin treatment effectiveness unit 302, specifically the processor 309. In some embodiments, the actuator-driven formula dispensing mechanism 306 includes a formula reservoir 313, a formula outlet 317 covered by a check valve 315, and an actuator 325. In some embodiments, the actuator is disposed within the formula reservoir 313. In some embodiments the formula reservoir is filled with a quantity of topical skin treatment formula 310A.

In some embodiments, the actuator 325 receives power from the power storage source 309. In some embodiments, the processor 309 regulates the operations and functions of the actuator 325. In some embodiments, the actuator (e.g., a piston) extends into the formula reservoir 313, increasing the pressure in the formula reservoir 313. The increase in pressure causes the formula 310B inside to flow out of the formula reservoir 313 through the outlet 317, as regulated by the check valve 315. In some embodiments, the processor 309 regulates the operations and functions of the check valve 315. In some embodiments, the rate of flow of the formula 310B can be adjusted by the processor 309.

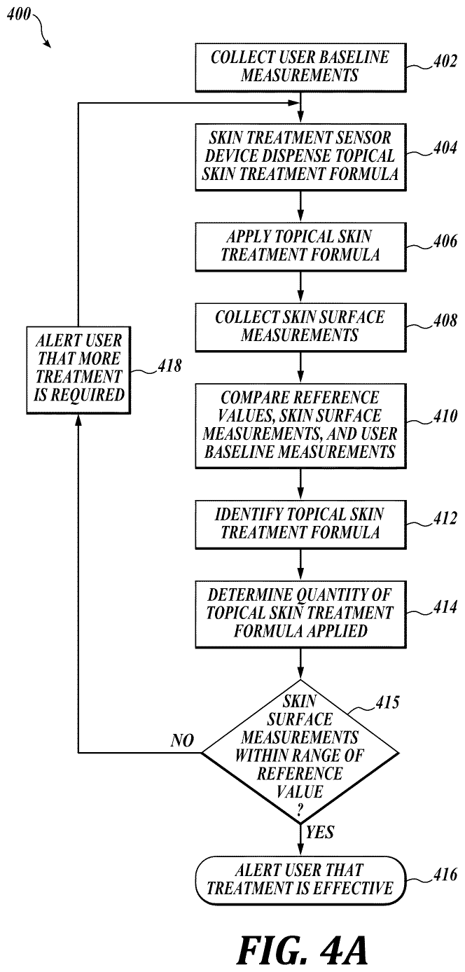

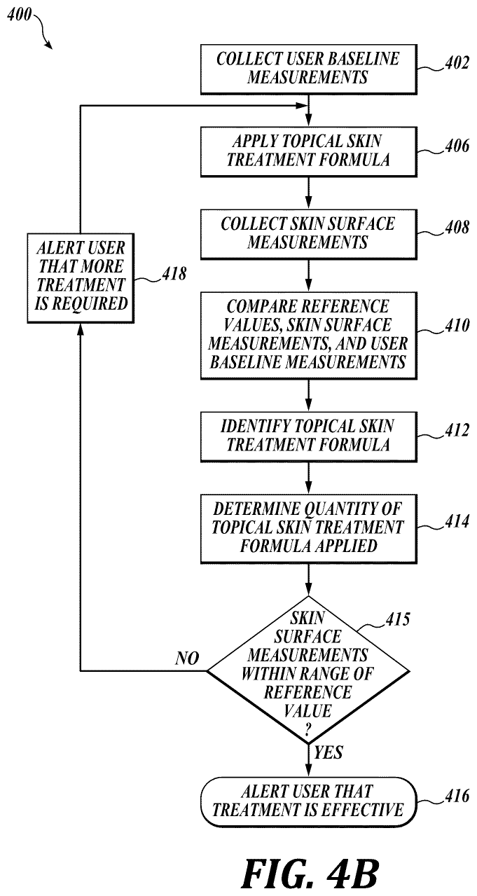

FIGS. 4A-4B are example methods for using a system for applying and analyzing a skin treatment formula with a skin treatment sensor device, in accordance with the present technology. It should be understood that components referred to in these figures are analogous to components identified in the system 100 described in FIG. 1. In different embodiments, the sample methods may operate without applying all the illustrated steps or may include the steps that are not illustrated in the flowchart.

FIG. 4A depicts an example method 400 for using a system for applying and analyzing a skin treatment formula with a skin sensor device where the device includes a formula dispensing mechanism. In process block 402, the user utilizes the skin treatment sensor device to collect user baseline measurements on the user's untreated skin surface. User baseline measurements may include skin resistivity, skin reflectivity, and skin temperature, which may be collected by an electroconductivity sensor, a spectroscopy sensor, and a temperature sensor, respectively. This process block need not be performed immediately prior to subsequent process blocks. In process block 404, the skin treatment sensor device dispenses at least one topical skin treatment formula to be applied to a user's skin surface. In some embodiments, the skin treatment sensor device includes a formula dispensing mechanism to perform this task, as described in FIGS. 3A-3B. In process block 406, the user applies the dispensed topical skin treatment formula to the skin surface that will be treated.

In process block 408, the user utilizes the skin treatment sensor device and its one or more sensors to collect skin surface measurements on the area of the skin surface where the topical skin treatment formula has been applied. Skin surface measurements may include skin resistivity, skin reflectivity, and skin temperature, which may be collected by an electroconductivity sensor, a spectroscopy sensor, and a temperature sensor, respectively.

In process block 410, the skin treatment sensor device may compare the skin surface measurement collected to the user baseline measurements collected in process block 402 and reference values stored in the device's memory. This comparison may be performed by the device's processor. These reference values may include known and quantifiable skin surface measurements that result from the unique characteristics of the topical skin treatment formulas. In process block 412, the skin treatment sensor device may identify the topical skin treatment formula applied to the skin surface. This may be performed by the device's processor, which utilizes the comparison conducted in the previous process block. Given a known relationship between the quantity of topical skin treatment formula applied and the skin surface measurement stored as a reference value in the device's memory unit, the processor may identify the formula applied by determining the impact of the formula on the user's baseline measurement. In process block 414, the skin treatment sensor device may determine the quantity of the identified topical skin treatment formula applied to the skin surface using the comparison conducted in process block 410. Given a known relationship between the quantity of topical skin treatment formula applied and the skin surface measurement stored as a reference value in the device's memory unit, the processor may determine the quantity of formula applied by determining the extent of the impact of the formula on the user's baseline measurement.

Process block 415 represents a decision point for the processor. In some embodiments, the reference values stored in the skin treatment sensor device's memory unit may include an ideal range or threshold value for a skin surface measurement, which may be compared in process block 410. If the skin surface measurements are within that ideal range of reference values or have reached a threshold reference value, the processor may determine that a sufficient amount of topical skin treatment formula has been applied to the user's skin surface and continue to process block 416. If the skin surface measurements are not within an ideal range of reference values or have not reached a threshold value, the processor may determine that the amount of topical skin treatment formula applied is not sufficient for ideal results and continue to process block 418.

In process block 416, the skin treatment sensor device alerts the user that the treatment is effective and directs the user to cease treatment using the user alert unit. In process block 418, the skin treatment sensor device alerts the user that the treatment is insufficient and directs the user to apply more of the topical skin treatment formula to the user's skin surface. Process block 416 then directs the user back to process block 404 and the method resumes from there. In some embodiments, the user alert unit includes a speaker. In such embodiments, the one or more notifications may be auditory and comprise one or more pre-recorded tones or, alternatively, a pre-recorded voice message. In some embodiments, the user alert unit includes a digital screen. In such embodiments, the one or more notifications may be visual and take the form of text displayed on the digital screen. In some embodiments, the user alert unit includes one or more LEDs. In such embodiments, the one or more notifications may be visual and take the form of one or more LEDs illuminating.

FIG. 4B depicts an example method 400 for using a system for applying and analyzing a skin treatment formula with a skin sensor device where the device does not include a formula dispensing mechanism. Each process block described is the same as described in FIG. 4A; however, process block 404—in which the skin treatment sensor device dispenses at least one topical skin treatment formula—does not occur according to this embodiment, as the device may lack the requisite formula dispensing mechanism. In such embodiments, the topical skin treatment formula may be dispensed directly by the user from another container, bottle, or reservoir.

It should be understood that method 400 should be interpreted as merely representative. In some embodiments, process blocks of this method may be performed simultaneously, sequentially, in a different order, or even omitted, without departing from the scope of this disclosure.

Throughout this specification, several terms of art are used. These terms are to take on their ordinary meaning in the art from which they come, unless specifically defined herein or the context of their use would clearly suggest otherwise. It should be noted that element names and symbols may be used interchangeably through this document (e.g., Si vs. silicon); however, both have identical meaning.

While illustrative embodiments have been illustrated and described, it will be appreciated that various changes can be made therein without departing from the spirit and scope of the invention.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

We claim:

1. A system for applying and analyzing a skin treatment formula, the system comprising:
at least one topical skin treatment formula configured for applying to a skin surface; and
a skin treatment sensor device comprising:
one or more skin sensors configured to collect a plurality of skin surface measurements;
a skin treatment effectiveness unit that is electrically coupled with the one or more skin sensors, the skin treatment effectiveness unit comprising:
a processor configured to identify a type and an amount of the at least one topical skin treatment formula applied to the skin surface based on the plurality of skin surface measurements;
a memory unit electrically coupled with the processor, the memory unit configured to store a plurality of reference values corresponding to a type and amount of the topical skin treatment formula applied to the skin surface and a plurality of user baseline measurements corresponding to the skin surface; and
a user alert unit electrically coupled with the processor, the user alert unit configured to present one or more notifications to a user.

2. The system of claim 1, wherein the one or more skin sensors comprise at least one of:
an electroconductivity sensor;
a spectroscopy sensor; and
a temperature sensor.

3. The system of claim 2, wherein the plurality of user baseline measurements, the plurality of skin surface measurements, and the plurality of reference values comprise at least one of skin resistivity, skin reflectivity, and skin temperature.

4. The system of claim 1, wherein the processor is configured for:
comparing the plurality of skin surface measurements with the plurality of reference values and the plurality of user baseline measurements; and
determining which of the one or more notification to present to the user based upon a comparison of the plurality of skin surface measurements, the plurality of reference values, and the plurality of user baseline measurements.

5. The system of claim 4, wherein the one or more notifications comprise:
a first notification directing the user to apply at least one skin treatment formula to the skin surface; and
a second notification directing the user to cease treatment.

6. The system of claim 1, wherein the skin treatment sensor device further comprises a power storage source configured to independently power the skin treatment sensor device.

7. The system of claim 1, wherein the skin treatment sensor device further comprises:
a formula dispensing mechanism, wherein the skin treatment effectiveness unit is electrically coupled with the formula dispensing mechanism.

8. A skin treatment sensor device comprising:
one or more skin sensors configured to collect a plurality of skin surface measurements;
a skin treatment effectiveness unit that is electrically coupled with the one or more sensors comprising:
a processor configured to identify a type and an amount of at least one topical skin treatment formula applied to a skin surface based on the plurality of skin surface measurements;
a memory unit electrically coupled with the processor, the memory unit configured to store a plurality of reference values corresponding to a type and amount of the topical skin treatment formula applied to the skin surface and a plurality of user baseline measurements corresponding to the skin surface; and
a user alert unit electrically coupled with the processor, the user alert unit configured to present one or more notifications to a user.

9. The device of claim 8, wherein the one or more skin sensors comprise at least one of:
an electroconductivity sensor;
a spectroscopy sensor; and
a temperature sensor.

10. The device of claim 9, wherein the plurality of user baseline measurements, the plurality of skin surface measurements, and the plurality of reference values comprise at least one of skin resistivity, skin reflectivity, and skin temperature.

11. The device of claim 7, wherein the processor is configured for:
comparing the plurality of skin surface measurements with the plurality of reference values and the plurality of user baseline measurements; and
determining which of the one or more notifications to present to the user based upon a comparison of the plurality of skin surface measurements, the plurality of reference values, and the plurality of user baseline measurements.

12. The device of claim 7, wherein the one or more notifications comprise:
a first notification directing the user to apply the at least one topical skin treatment formulas to the skin surface; and
a second notification directing the user to cease treatment.

13. The device of claim 7, further comprising:
a formula dispensing mechanism, wherein the skin treatment effectiveness unit is electrically coupled with the formula dispensing mechanism.

14. The device of claim 7, wherein the skin treatment sensor device further comprises a power storage source configured to provide power to the skin treatment sensor device.

15. A method for determining the effectiveness of a skin treatment, the method comprising:
collecting a plurality of user baseline measurements;
applying at least one topical skin treatment formulas to a skin surface;
identifying, by a skin treatment sensor device, the at least one topical skin treatment formulas applied to the skin surface;
collecting a plurality of skin surface measurements;
comparing a plurality of reference values corresponding to a type and amount of the topical skin treatment formula applied to the skin surface, the plurality of skin surface measurements, and the plurality of user baseline measurements corresponding to the skin surface; and
alerting a user of the effectiveness of the skin treatment.

16. The method of claim 15, wherein the plurality of user baseline measurements, the plurality of skin surface measurements, and the plurality of reference values comprise skin resistivity, skin reflectivity, and skin temperature.

17. The method of claim 15, wherein the skin treatment sensor device comprises:
one or more skin sensors configured to collect a plurality of skin surface measurements;
a skin treatment effectiveness unit that is electrically coupled with the one or more sensors comprising:
a processor configured to identify a type and an amount of at least one topical skin treatment formula applied to the skin surface based on the plurality of skin surface measurements;
a memory unit electrically coupled with the processor, the memory unit configured to store a plurality of reference values corresponding to the type and amount of the topical skin treatment formula and a plurality of user baseline measurements corresponding to the skin surface; and
a user alert unit electrically coupled with the processor, the user alert unit configured to present one or more notifications to the user.

18. The method of claim 17, wherein the processor is configured for:
comparing the plurality of skin surface measurements with the plurality of reference values and the plurality of user baseline measurements; and
determining which of the one or more notifications to present to the user based upon a comparison of the plurality of skin surface measurements, the plurality of reference values, and the plurality of user baseline measurements.

19. The method of claim 17, wherein the one or more notifications comprise:
a first notification directing the user to apply the at least one topical skin treatment formulas to the skin surface; and
a second notification directing the user to cease treatment.

20. The method of claim 15, further comprising:
dispensing, by the skin treatment sensor device, a topical skin treatment formula.
